(19) United States
(12) Patent Application Publication
(10) Pub. No.: US 2008/0057100 A1
(43) Pub. Date: Mar. 6, 2008

(54) Implantable device for delivery of therapeutic agents

(76) Inventors: Michael S. Williams, Santa Rosa, CA (US); Wenda Carlyle, Newton, CT (US)

Correspondence Address: DEANNA J. SHIRLEY, 3418 BALDWIN WAY, SANTA ROSA, CA, 95403, US

(21) Appl. No.: 11/978,047

(22) Filed: Oct. 25, 2007

Related U.S. Application Data
(63) Continuation-in-part of application No. 11/150,374, filed on Jun. 11, 2005.
(60) Provisional application No. 60/582,184, filed on Jun. 22, 2004.

(51) Int. Cl. A61F 2/02; A61K 35/12

(52) U.S. Cl. 424/423; 424/93.7

(57) ABSTRACT

A percutaneously implantable device for the treatment of a cardiac condition or other disease is disclosed herein, the device capable of delivery and maintenance of a therapeutic scaffold. A therapeutic scaffold may comprise viable tissue to impart or restore normal cardiac function, or other therapeutic agent for the treatment of disease or injury. Viable tissue may comprise a pacemaker gene or other genes intended to impart a pacemaker function to either host tissue or transplanted tissue, or both. Further, a device according to the invention may be used for the implantation and maintenance of viable tissue to induce or enhance muscle contraction of a subject for the treatment of a disease or disorder.

### RELATED APPLICATIONS

This application is related to and claims the benefit of the priority date of Provisional U.S. Patent Application Ser. No. 60/582,184 titled “Implantable Chamber for Biological Induction or Enhancement of Muscle Contraction”, filed Jun. 22, 2004, by Williams, and is a continuation in part of U.S. patent application Ser. No. 11/150,374, titled “Implantable Chamber for Biological Induction or Enhancement of Muscle Contraction”, filed Jun. 11, 2005 by Williams.

### FIELD OF THE INVENTION

The invention herein is related to implantable medical devices and more specifically to devices and methods for delivery of one or more therapeutic scaffolds. Devices, scaffolds, and methods for administering long term therapies, including, for example, inducing, restoring or enhancing muscle contraction are disclosed. In a specific example, the invention is an artificial sinoatrial node or atrioventricular node of the mammalian heart. In another example, the invention is an intraseptal implant comprising a therapeutic agent packaged in a polymer matrix, which releases the therapeutic agent over an extended period of time. Further, the invention relates to a percutaneously implantable chamber for delivery and maintenance of, for example, a viable tissue scaffold for the conduction of the pacemaker current from the cells within the tissue scaffold to the endogenous cardiac myocytes of a subject.

### BACKGROUND OF THE INVENTION

Specialized cardiac conducting tissue and the myocardium maintain an intrinsic rhythm in the healthy mammalian heart. The heart's rate is mediated through the autonomic nervous system which operates on a small mass of muscle cells called the sinoatrial (SA) node, which is located on the right atrium of the heart. An electrical signal generated by this structure causes the atria of the heart to contract. Contraction of the atria forces blood into the ventricles of the heart. The signal from the SA node is propagated to the ventricles through a structure called the atrioventricular (AV) node, an area of specialized tissue located on the interatrial septum, and close to the tricuspid valve, after a brief delay. The signal from the AV node, traveling therethrough as the path of least resistance to the ventricles, causes the ventricles to contract, forcing the blood throughout the body.

Many forms of heart disease impair the function of the SA and AV nodes and their associated conductive tissues, and can lead to abnormalities of the heart rhythm. These abnormalities, generally referred to as arrhythmias, potentially lead to substantial patient discomfort or even death. Morbidity and mortality from such problems is significant to the public health. In the United States alone for example, cardiac arrest accounts for 220,000 deaths per year, possibly more than 10% of total American deaths.

Implantable medical devices developed for the management of cardiac rhythm, referred to herein as pacemakers, have been helpful and even life saving for a substantial number of patients suffering cardiac arrhythmia A typical pacemaker includes a pulse generator, a power source, a pacing lead, electronic circuitry, and a programmer. The pulse generator sends electrical stimulation pulses through the pacing leads to stimulate the heart to beat in a controlled rhythm. Advanced pacemakers may include physiological sensors in order to provide pacing that is responsive to a patient's level of activity and other varying physiological demands. However, such devices are unable to perform the complex physiological functions of normal, healthy cardiac cells. Additionally, such advances require additional circuitry and increase the demands of the power source, thereby competing with the desire for smaller, affordable and longer lasting devices. Drawbacks of all pacemakers include the need for maintenance and power source replacement.

It is therefore desirable to provide a device and method for increasing and/or restoring the physiological function of the natural cardiac pacemaker cells and the myocardium. In addition to being maintenance free, such cells will be naturally responsive to emotional and hormonal changes and varied activity levels of a patient, and are a curative solution to the disease state, rather than a palliative measure.

Some advances have been made in the development of biological cell lines and tissue constructs that record a pacemaker current and consequently are potentially able to perform the cardiac pacemaker function Researchers have demonstrated that cardiac tissue engineered constructs transplanted into rat hearts will form functional gap junctions with native cardiac cells and the transplanted tissue will survive for the lifetime of the animal (See Choi et al., “Cardiac conduction through engineered tissue”, Am J Path 169 (1): 72-85 (2006).

Such advances also hold some promise for advances in the treatment of other disorders related to muscle contraction, including, for example, stress incontinence. Further, the technology may be used in targeted muscle contraction to regulate food intake for the treatment of obesity. However, there remains a need in the art for a device and a method by which to deliver such cells and/or tissue constructs to a desired treatment site in a minimally invasive manner. Further, there remains a need for preventing the migration of cells from the desired site following delivery. If the cells or tissue scaffolds are retained in order to function at the target site, the retention device must be suitable for tissue function and for the continued viability of cells. For example, the device must permit the entry and exit of materials necessary for and resulting from cellular respiration, such as, for example, oxygen, nutrients, electrolytes, carbon dioxide, and lactic acid. It is also desirable that the device itself not provoke an excessive immune response.

Still further, the means of retention must not prohibit the formation of cell-cell gap junctions between the implanted cells and the endogenous cells. The device must permit the electrical conductivity of the pacemaker current generated by the cells and/or tissue constructs to the endogenous cardiac myocytes. The device's surfaces must be non-fouling, and prevent encapsulation by overgrowth of cells, or, in the alternative, promote endogenous cell growth and neovascularization.

Other diseases and injury, whether of the heart or other organ systems, require sustained administration of a therapeutic agent. Many therapeutic agents that are commonly delivered orally or as inhalants are subject to the drawbacks of erratic absorption, disruption of a patient's digestive or other processes as well as other undesirable side effects that are the result of the method of administration. Additionally, other therapeutic agents must currently be delivered intravenously in order to be effective, with the attendant ongoing required medical care and other inconvenience.

Numerous therapeutic agents hold promise of clinical benefit if delivered via an implanted intraseptal device for prolonged, and potentially very long term periods of time, for the treatment of various forms of heart disease and other disease or injury. In addition, the potential drawbacks of oral, nasal or intravenous delivery may be avoided. Antithrombotics, anticoagulants, antiplatelets, antiinflammatories, antiinfectives, antifibrotics, antineoplastics, antivirals, immunosuppressants, antihypertensives, anticholesterols, analgesics, anticonvulsants, antidiabetics, antipsychotics, hormones, cardioprotectives and antibiotics are some examples of therapies that potentially may be delivered via an intraseptal device. In addition, there is also a need in the art for reliable sustained delivery of therapies for diseases such as Parkinson's, epilepsy and various blood disorders. The abilities to manage sustained delivery, to increase convenience to a patient and to improve compliance are also needed in the art.

### SUMMARY OF THE INVENTION

An implantable device for the delivery and maintenance of a therapeutic scaffold, comprising one or more anchors is disclosed. The anchors are configured to retrievably secure the device within a septal wall of the heart of a subject, and the scaffold may be exchangeable and/or refillable. The therapeutic scaffold may comprise a viable tissue prepared to impart a pacemaker function to the heart of a subject. Alternatively, the therapeutic scaffold may include one or more pharmaceutical, chemical, biological or radiological agents. The scaffold one or more projections thereby increasing the surface area of the scaffold.

The device may also have a frame configured to retain the therapeutic scaffold. The anchor or anchors may or may not be integral with the frame. A device according to the invention may have a delivery configuration and a deployed configuration and may be delivered percutaneously to a subject. It may be constructed with one or more shape memory materials, and may have a selectively permeable membrane. The device may deliver a tissue scaffold to a subject in order to induce or enhance muscle contraction.

via cell-cell gap junction formationA method for the minimally invasive treatment of a disease or condition is disclosed, the method comprising the steps of providing a device comprising one or more therapeutic scaffolds; the device may comprise a delivery configuration and a deployed configuration; accessing the right or left atrium, or right or left right ventricle of a subject; penetrating the atrial or ventricular septal wall; delivering the device to the atrial or ventricular septal wall; and deploying the device within the atrial or ventricular septal wall.

The device may have one or more anchors, means for retaining the therapeutic scaffold, and the method may have the added step of deploying the anchors for securing the chamber within the atrial or ventricular septal wall. The therapeutic scaffold may comprise a viable tissue prepared to impart a pacemaker function to the heart of a subject. Alternatively, the therapeutic scaffold may comprise one or more pharmaceutical, chemical, biological or radiological agents. The device may comprise a selectively permeable membrane, and may comprise one or more shape memory materials.

The device may be retrievably implantable, and the therapeutic scaffolds may be exchangeable and/or refillable. The therapeutic scaffold may comprise viable, electrically conductive tissue to induce or enhance muscle contraction in a subject, and the method may be used to treat a cardiac rhythm disorder. The method may be performed percutaneously.

### BRIEF DESCRIPTION OF THE DRAWINGS

FIG. 1 illustrates a frontal cross sectional view of the human heart and related vasculature.

FIG. 2 illustrates the anatomical region of FIG. 1 into which a means of delivery of an embodiment according to the invention has been introduced.

FIG. 3 illustrates in larger detail a frontal cross sectional view of the human heart and related vasculature and the introduction of a means of delivery of an embodiment according to the invention.

FIGS. 4-11 illustrate, in perspective cutaway view, a selection of successive steps of deployment of an embodiment according to the invention within a septal wall of the heart of a subject.

FIG. 12 illustrates a perspective view of an embodiment according to the invention in a deployed configuration.

FIG. 13 illustrates side view of the embodiment of FIG. 13 in a deployed configuration.

FIG. 14 illustrates an “exploded” perspective view of the embodiment of FIGS. 12 and 13.

### DETAILED DESCRIPTION OF THE INVENTION

A “self-expanding” device has the ability to revert readily from a reduced profile configuration to a larger profile configuration in the absence of a restraint upon the device that maintains the device in the reduced profile configuration.

“Expandable” refers to a device that comprises a reduced profile configuration and an expanded profile configuration.

“Expansion ratio” refers to the percentage increase in diameter of a device following conversion of the device from its reduced profile configuration to its expanded profile configuration.

“Elasticity” refers to the ability of a material to repeatedly undergo significant tensile stress and strain, and/or compression stress and strain, and return to its original configuration.

A “switching segment” comprises a transition temperature and is responsible for the shape memory polymer's ability to fix a temporary shape.

A “thermoplastic elastomer” is a shape memory polymer comprising crosslinks that are predominantly physical crosslinks.

A “thermoset” is a shape memory polymer comprising a large number of crosslinks that are covalent bonds.

Although a device according to the invention may be manufactured from a suitable metal, it may alternatively be manufactured from a polymer, such as, for example, expanded polytetrafluoroethylene (ePTFE) which may vary in porosity. A device comprising polymeric materials has the advantage of compatibility with magnetic resonance imaging, potentially a long term clinical benefit. Further, if the more conventional diagnostic tools employing fluoroscopic visualization continue as the technique of choice for delivery and monitoring, radiopacity can be readily conferred upon polymeric materials. The use of polymeric materials in the fabrication of devices confers the advantages of improved flexibility, compliance and conformability, enhancing percutaneous delivery.

Examples of conductive polymers include, but are not limited to: polyaniline, polythiophene and their derivatives, and others.

Although the invention herein is not limited as such, portions of some embodiments of the invention comprise materials that are bioerodible. “Erodible” refers to the ability of a material to maintain its structural integrity for a desired period of time, and thereafter gradually undergo any of numerous processes whereby the material substantially loses tensile strength and mass. Examples of such processes comprise hydrolysis, enzymatic and non-enzymatic degradation, oxidation, enzymatically-assisted oxidation, and others, thus including bioresorption, dissolution, and mechanical degradation upon interaction with a physiological environment into components that the patient's tissue can absorb, metabolize, respire, and/or excrete.

Polymer chains are cleaved by hydrolysis and are eliminated from the body through the Krebs cycle, primarily as carbon dioxide and in urine. “Erodible” and “degradable” are intended to be used interchangeably herein.

“Embedded” agents are set upon and/or within a mass of material by any suitable means including, but not limited to, combining the agent with the material while the material (such as, for example, a polymer) is in solution, combining the agent with the material when the material is heated near or above its melting temperature, affixing the agent to the surface of the material, and others.

“Balloon expandable” refers to a device that comprises a reduced profile configuration and an expanded profile configuration, and undergoes a transition from the reduced configuration to the expanded configuration via the outward radial force of a balloon expanded by any suitable inflation medium.

The term “balloon assisted” refers to a self-expanding device the final deployment of which is facilitated by an expanded balloon.

As used herein, a device is “implanted” if it is placed within the body to remain for any length of time following the conclusion of the procedure to place the device within the body.

The term “diffusion coefficient” refers to the rate by which a substance elutes, or is released either passively or actively from a substrate.

Unless specified, suitable means of manufacture and assembly of a device according to the invention may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is substantially recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, and others. Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions. Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)ₛ, especially poly(.beta-hydroxybutyrate), poly(3-hydroxyoctanoate) and poly(3-hydroxyfatty acids).

Representative natural erodible polymer segments or polymers include polysaccharides such as alginate, dextran, cellulose, collagen, and chemical derivatives thereof (substitutions, additions of chemical groups, for example, alkyl, alkylene, hydroxylations, oxidations, and other modifications routinely made by those skilled in the art), and proteins such as albumin, zein and copolymers and blends thereof, alone or in combination with synthetic polymers.

Suitable synthetic polymer blocks include polyphosphazenes, poly(vinyl alcohols), polyamides, polyester amides, poly(amino acid)s, synthetic poly(amino acids), polyanhydrides, polycarbonates, polyacrylates, polyalkylenes, polyacrylamides, polyalkylene glycols, polyalkylene oxides, polyalkylene terephthalates, polyortho esters, polyvinyl ethers, polyvinyl esters, polyvinyl halides, polyvinylpyrrolidone, polyesters, polylactides, polyglycolides, polysiloxanes, polyurethanes and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

Examples of synthetic degradable polymer segments or polymers include polyhydroxy acids, polylactides, polyglycolides and copolymers thereof, poly(ethylene terephthalate), poly(hydroxybutyric acid), poly(hydroxyvaleric acid), poly[lactide-co-(epsilon-caprolactone)], poly[glycolide-co-(epsilon-caprolactone)], polycarbonates, poly-(epsilon caprolactone) poly(pseudo amino acids), poly(amino acids), poly(hydroxyalkanoate)s, polyanhydrides, polyortho esters, and blends and copolymers thereof.

Rapidly erodible polymers such as poly(lactide-co-glycolide)s, polyanhydrides, and polyorthoesters, which have carboxyl groups exposed on the external surface as the smooth surface of the polymer erodes, also can be used. In addition, polymers containing labile bonds, such as polyanhydrides and polyesters, are well known for their hydrolytic reactivity. Their hydrolytic degradation rates can generally be altered by simple changes in the polymer backbone and their sequence structure.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly(ethylene terephthalate), poly(vinyl acetate), and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

An additional advantage of polymers includes the ability to control and modify properties of the polymers through the use of a variety of techniques. According to the invention, optimal ratios of combined polymers, optimal configuration of polymers synthesized to exhibit predictable rates of erosion, and optimal processing have been found to achieve highly desired properties not typically found in polymers. In general, erosion of a polymer will progress at a known range of rates. Environmental factors such as PH, temperature, tissue or blood interaction and other factors such as structural design of the device all impact the degradation rate of erodible polymers. Depending upon the desired performance characteristics of a device, in some cases it may be desirable to either “program in” a desired rate of erosion, or desired cycle of varied rates of erosion, to initiate on-demand erosion of a device, or to have a set of desired mechanical properties or to function in a desired manner for a period of time, and an alternative set of desired mechanical properties for a second period of time. For example, it may be desirable for the device to deliver a therapeutic substance under particular conditions and/or during a particular time period.

According to the invention, a polymer may be tailored to erode rapidly during one phase, such as, for example, a therapy delivery phase, followed by a period of time during which the polymer erodes at a slower rate. Such a time period of slower erosion may be followed by a second drug delivery phase during which the polymer again erodes rapidly. Similarly, a polymer may be tailored to erode on demand, upon the introduction of a stimulus such as increase in temperature, exposure to radiation, and/or others. Any number of combinations of desired phases is possible according to the invention.

The rate of erosion of a polymer may be controlled by one or more of several techniques. An example of such a technique includes the incorporation of an agent or substance that acts as a catalyst of degradation upon exposure to a stimulus. Examples of such agents or substances include, but are not limited to, sensitizers, dissolution inhibitors, biochemically active additives, thermal, light, electromagnetic radiation, or enzyme-activated catalysts, or some combination of the foregoing. Examples of sensitizers include, but are not limited to photoacid generators (PAGs), dissolution inhibitors, and radiosensitizers. Examples of biochemically active additives include, but are not limited to, lipids or peptides susceptible to degradation by specific enzymes. Further, one or more layers of polymer comprising one of the foregoing agents may alternate with a layer of polymer that does not comprise such an agent, or is tailored to erode at a different rate or upon the introduction of an alternate stimulus. More specific examples of the foregoing in set forth in provisional U.S. Patent Application Ser. No. 60/633,494, and are incorporated as if set forth fully herein.

According to another aspect of the invention, surface treatment including, but not limited to removal of impurities and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”). Compressing a gas normally causes a phase separation and the appearance of a separate liquid phase. However, all gases have a critical temperature above which the gas cannot be liquefied by increasing pressure, and a critical pressure or pressure which is necessary to liquefy the gas at the critical temperature. For example, carbon dioxide in its supercritical state exists as a form of matter in which its liquid and gaseous states are indistinguishable from one another. For carbon dioxide, the critical temperature is about 31 degrees C. (88 degrees D) and the critical pressure is about 73 atmospheres or about 1070 psi.

The term “supercritical carbon dioxide” as used herein refers to carbon dioxide at a temperature greater than about 31 degrees C. and a pressure greater than about 1070 psi. Liquid carbon dioxide may be obtained at temperatures of from about −15 degrees C. to about −55 degrees C. and pressures of from about 77 psi to about 335 psi. One or more solvents and blends thereof may optionally be included in the carbon dioxide. Illustrative solvents include, but are not limited to, tetrafluoroisopropanol, chloroform, tetrahydrofuran, cyclohexane, and methylene chloride. Such solvents are typically included in an amount, by weight, of up to about 20%.

In general, carbon dioxide may be used to effectively lower the glass transition temperature of a polymeric material to facilitate the infusion of pharmacological agent(s) into the polymeric material. Such agents include but are not limited to hydrophobic agents, hydrophilic agents and agents in particulate form. For example, following fabrication, a device and a hydrophobic pharmacological agent may be immersed in supercritical carbon dioxide. The supercritical carbon dioxide “plasticizes” the polymeric material, that is, it allows the polymeric material to soften at a lower temperature, and facilitates the infusion of the pharmacological agent into the polymeric device or polymeric coating of a stent at a temperature that is less likely to alter and/or damage the pharmacological agent.

As an additional example, a device and a hydrophilic pharmacological agent can be immersed in water with an overlying carbon dioxide “blanket”. The hydrophilic pharmacological agent enters solution in the water, and the carbon dioxide “plasticizes” the polymeric material, as described above, and thereby facilitates the infusion of the pharmacological agent into a polymeric device or a polymeric coating of a device.

As yet another example, carbon dioxide may be used to “tackify”, or render more fluent and adherent a polymeric device or a polymeric coating on a device to facilitate the application of a pharmacological agent thereto in a dry, micronized form.

A membrane-forming polymer, selected for its ability to allow the diffusion of the pharmacological agent therethrough, may then applied in a layer over the device. Following curing by suitable means, a membrane that permits diffusion of the pharmacological agent over a predetermined time period forms. Surface treatment for the removal of impurities or the incorporation of a therapeutic substance are more fully set forth in commonly owned U.S. patent application Ser. Nos. 10/662,621 and 10/662,757, which are hereby incorporated in their entirety as if set forth fully herein.

Objectives of therapeutic substances coating a device according to the invention include reducing the adhesion and aggregation of platelets on the surface of the implant, preventing an inflammatory or immunological reaction to the device, augmenting a neovascular response to improve perfusion of blood and nutrients to the device, and/or the homing of progenitor cells to the device or surrounding area. At the site of implantation, objectives may include to block the expression of growth factors and their receptors; develop competitive antagonists of growth factors, interfere with the receptor signaling in the responsive cell, promote an inhibitor of smooth muscle proliferation Anitplatelets, anticoagulants, antineoplastics, antifibrins, enzymes and enzyme inhibitors, antimitotics, antimetabolites, anti-inflammatories, antithrombins, antiproliferatives, antibiotics, anti-angiogenesis factors, pro-angiogenic factors, specific growth factors and others may be suitable.

“Cells” may be derived from adult mesenchymal stem cells, but may alternatively be embryonic stem cells, skeletal myoblasts, fetal cardiomyocytes, smooth muscle cells, bone marrow derived stromal and hematopoietic stem cells, or any cells suitable for the expression of one or more pacemaker genes. Autologous myoblasts or bone marrow derived stem cells may be less likely to provoke immunogenic response to the implanted scaffold. If the cells have been encoded with a desirable gene, it may be according to any suitable method including, but not limited to, electroporation, transfer through liposomes, a plasmid, a viral vector, dendrimers, cationic polymers, nanohydrogels, nanoparticles, crosslinked micelles, cell-penetrating peptides, cell targeting peptides or other suitable method. Said cells may be terminally differentiated and/or terminally quiescent. The cells may be autograft, allograft, xenograft, or some combination thereof.

“Pacemaker gene” may include any one of the genes that encode one or more of the proteins or subunits that play a role in regulating heart rate, and/or imposes pacemaker function on the atria, or any gene selected via acceptable means known in the art for the ability to confer pacemaker function on cells. Proteins or subunits that play a role in regulating heart rate include, but are not limited to, any of the family of hyperpolarization activated cyclic nucleotide gated (HCN) ion channels, Kir3.1/3.4, minimal potassium channel proteins or minimal potassium channel related peptides. Expression of pacemaker genes in stem cells has been reported and pacemaker current recorded from such cells in, for example, U.S. Patent Application Publication No. 2002/0187948, which is incorporated by reference herein in its entirety. Genes that have been recently shown to confer pacemaker activity on the heart include, but are not limited to, Tbx3. (See Hoogars et al., Genes and Dev 21: 1098-1112 (2007), which is incorporated herein as if included in its entirety.)

“Therapeutic agent” includes any material capable of action in, on or against a biological subject; most often the administration of a therapeutic agent will be with the intention of, but is not limited to, ameliorating disease or injury in a subject. Therapeutic agent may include, but is not limited to, viable biological tissue, cells, genes, fluid, or other material, as well as pharmaceutical or radiological preparation; antiplatelets, anticoagulants, antineoplastics, antifibrotics, hormones, enzymes and enzyme inhibitors, antimitotics, antimetabolites, anti-inflammatories, antithrombins, anticholesterols, cardioprotectives, antihypertensives, antivirals, antiproliferatives, antibiotics, immunosuppressants, antipsychotics, antidiabetics, analgesics, anti-angiogenesis factors, and other suitable agents.

A “therapeutic scaffold”, sometimes referred to as a “scaffold” herein, is any construct prepared utilizing suitable means to encase or give structure to a therapeutic agent for delivery of therapy over a desired period of time following implantation in a subject. A therapeutic scaffold and may include, for example, a viable tissue construct, or, as another example, a structure in which a pharmaceutical agent is suspended or encased within a polymer matrix. A scaffold may or may not be enclosed by a selectively porous membrane, and may also be a transvascularly refillable reservoir of therapeutic agent.

Therapeutic scaffolds comprising viable tissue most often are biocompatible, three-dimensional, collagen-based constructs containing myogenic precursor cells, or myoblasts, such as those described in American Journal of Pathology, Jul. 2006, Vol. 169, No. 1, pages 72-85, which is incorporated as if set forth fully herein.

Tissue scaffolds may comprise synthetic or biological materials or both Suitable examples include, but are not limited to porous alginate scaffolds, as described by Leor J et al. in “Bioengineered cardiac grafts; A new approach to repair the infarcted myocardium?” Cir 102 [suppl III] III-56-III-61, (2000); polyglycolic acid scaffolds, as described by Carrier R L et al. in “Cardiac tissue engineering: cell seeding, cultivation parameters, and tissue construct characterization”, Biotechnol Bioeng 64 (5): 580-589 (1999); collagen, as described by Kofidis et al. in “Distinct cell-to-fiber junctions are critical for the establishment of cardiotypical phenotype in a 3D bioartificial environment”, Med Eng Physics 26: 157-163 (2004); or collagen/Matrigel® combinations, as described by Zimmerman et al. in “Engineered heart tissue for regeneration of diseased hearts”, Biomaterials 25: 1639-1647 (2004); all of which are incorporated as if set forth fully herein. Developing cardiac constructs will most often desirably undergo in vitro mechanical stimulation and cell preconditioning during development of the tissue engineered composite, as described by Gonen-Wadmany et al. in “Controlling the cellular organization of tissue-engineered cardiac constructs”, Ann N Y Acad Sci 1015: 299-311 (2004), in order to provide cells with a three-dimensional environment and the correct biomechanical signals to orient myofibrils and establish structural adhesions with matrix proteins and electrical connectivity between cells via gap junctions. Tissue scaffolds may be cultured and grown in separate “trays” which may be stacked as multiple scaffolds to increase volume of prepared tissue.

Therapeutic scaffolds incorporating pharmaceutical or other active agents most often are biocompatible, three dimensional structures and may be a polymer matrix or membrane within which an agent is encased, enclosed, suspended or otherwise incorporated.

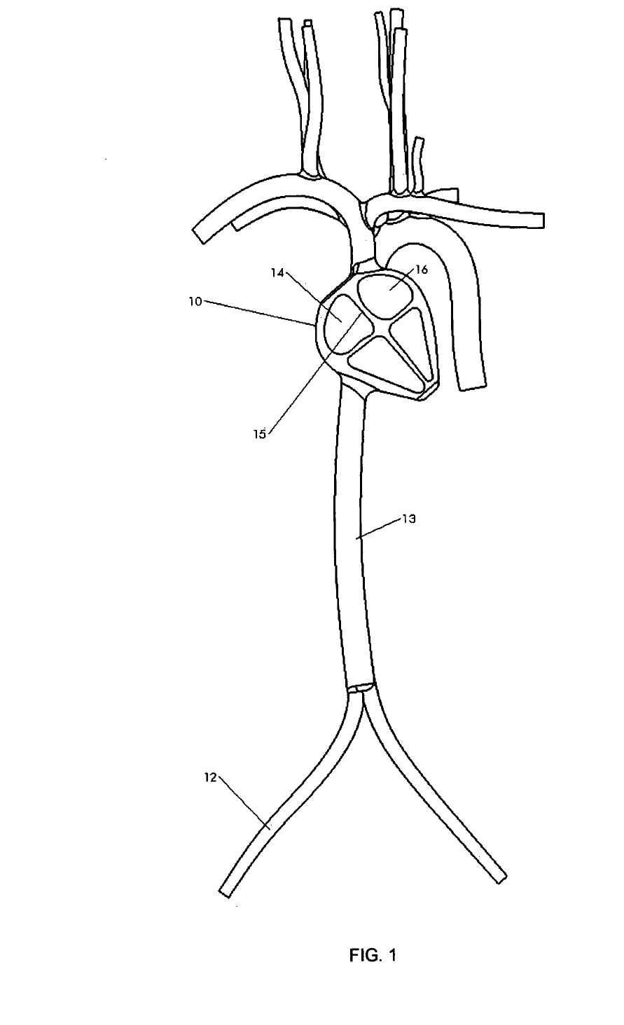

FIG. 1 illustrates an area of anatomical interest for employing a device and method according to the invention. In order to illustrate percutaneous delivery and deployment of a device according to the invention, FIG. 1 depicts a frontal view of human heart 10 and related vasculature, including right femoral vein 12, inferior vena cava 13, right atrium 14, interatrial septum 15, and left atrium 16.

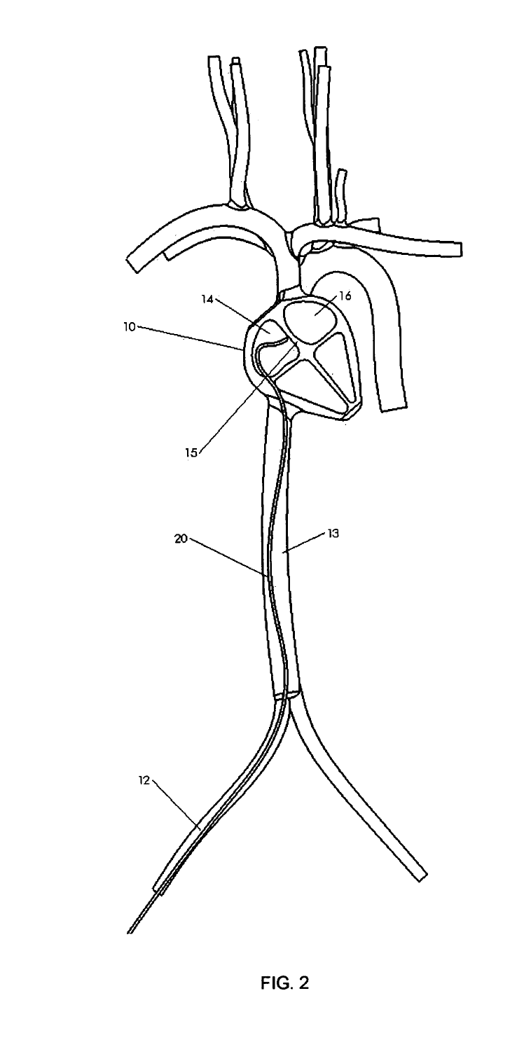

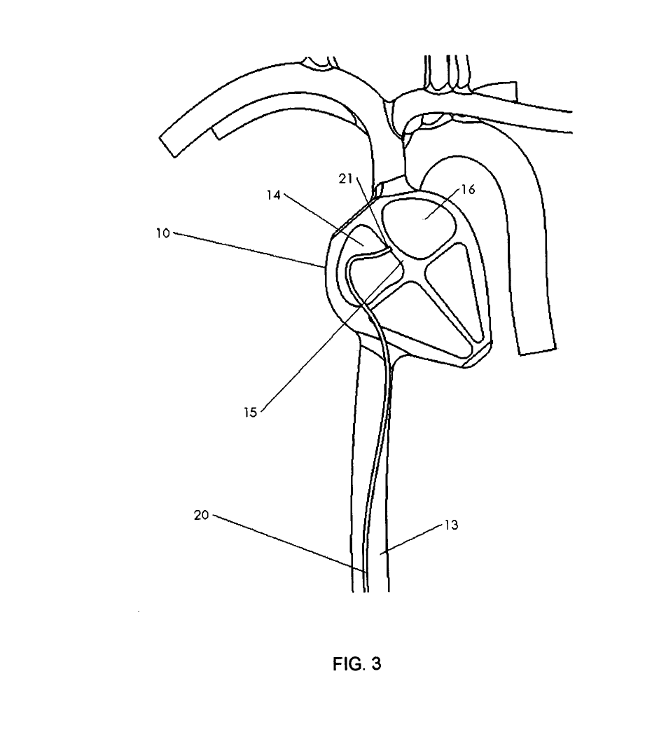

FIG. 2 illustrates the anatomical area of interest of FIG. 1 into which access catheter 20 has been introduced. The introduction may be achieved, for example, via an incision to access right femoral vein 12, which together with inferior vena cava defines a path to right atrium 14. Accordingly, as illustrated in FIG. 2, and in larger detail in FIG. 3, distal end 21 of access catheter 20 has been tracked into femoral vein 12, through inferior vena cava 13, into right atrium 14, and through interatrial septum 15 via any suitable cutting or piercing means, in order to permit simultaneous or subsequent delivery, implantation and deployment of a device according to the invention to the region of the AV node. (Alternatively, access catheter may be tracked further through the tricuspid valve and into the right ventricle. The ventricular septal wall may then be penetrated, in order to deploy a device therein. Other conceivable paths permit delivery and deployment of a device to alternative target sites and are also in accordance with the invention disclosed herein.) As described more fully below, an embodiment according to the invention may be delivered to the interatrial septum following the path of access catheter 20 illustrated in FIGS. 1-3.

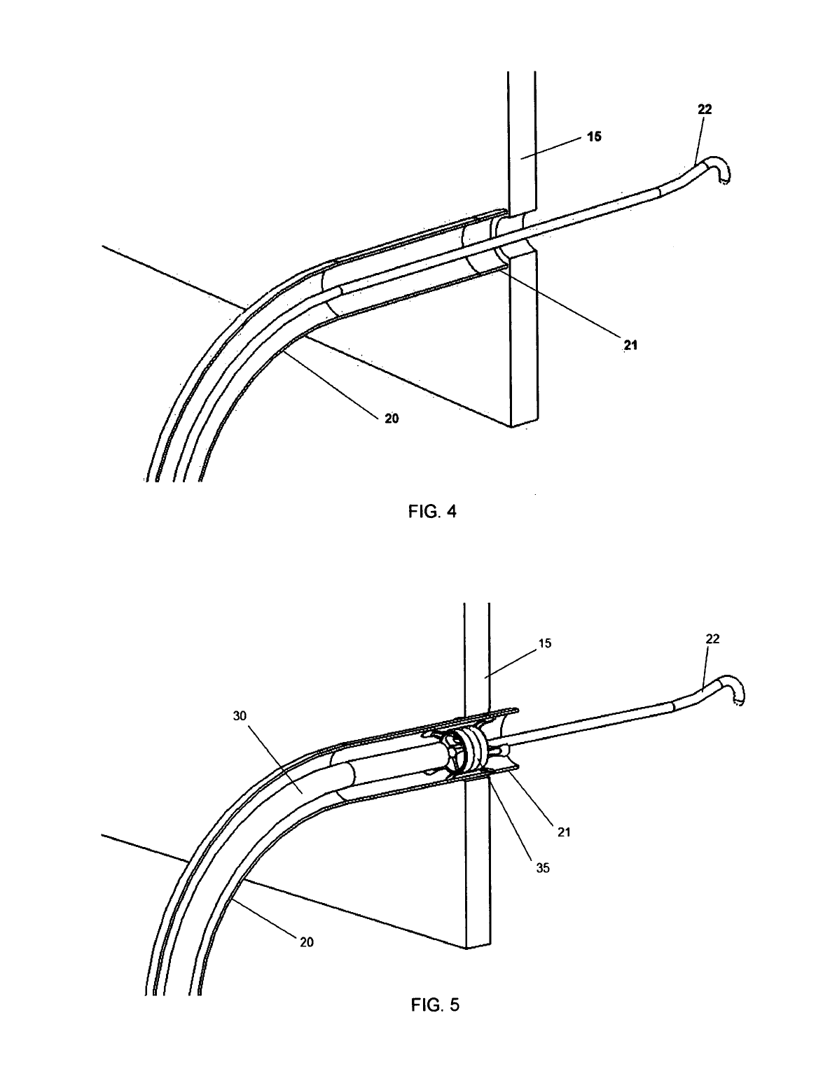

Selected steps within a series of steps to deliver and deploy a device according to the invention can be described with additional illustration provided beginning with FIG. 4 with emphasis on the method's and device's relation to the interatrial septum 15. In an exemplary preparatory step, as illustrated in perspective in FIG. 4 in cutaway mode, distal end 21 of access catheter 20, has penetrated (via suitable means) and been positioned through interatrial septum 15. Guide 22 extends through interatrial septum 15 into left atrium 16 (not pictured in FIG. 4.)

Subsequently, as shown in FIG. 5, delivery catheter 30 has been introduced via access catheter 20. Delivery catheter 30 carries implant 35 which is within its delivery configuration. A delivery configuration may comprise, for example, a reduced profile configuration in which implant 35 is releasably constrained. In addition to or in the alternative, a device's anchors (described more fully below) may be releasably constrained within a delivery configurations. Implant 35 may thereby be delivered percutaneously via delivery catheter 30. (Implant 35 may alternatively be delivered to the ventricular septal wall or other target site.) Delivery catheter 30 carries implant 35 which contains therapeutic scaffolds 36, 37 and 38. (Implant 35 may alternatively contain a smaller or greater number of therapeutic scaffolds, which may be of alternative suitable sizes, shapes and dimensions than those illustrated in FIG. 5.)

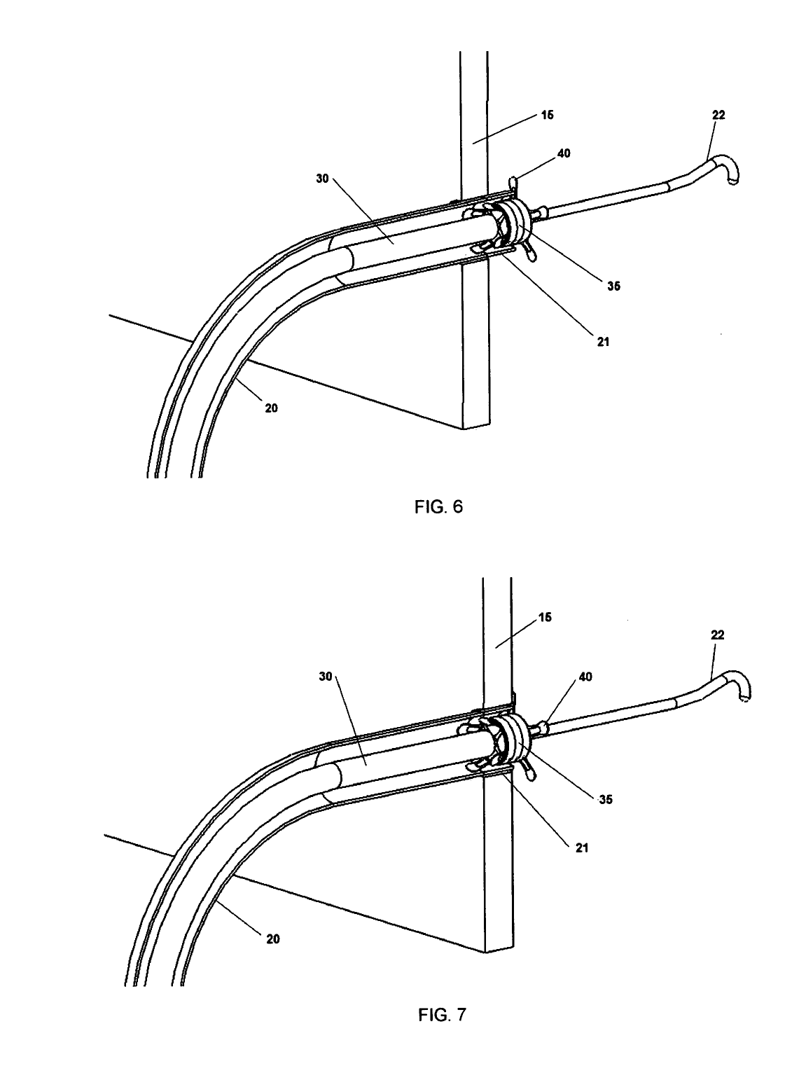

FIG. 6 illustrates a subsequent step in which distal end 21 of access catheter 20 has been tracked over guide 22 into left atrium 16. Once beyond interatrial septum 15, first end anchors 40, which may comprise, for example, stainless steel, or a shape memory material such as nickel titanium or a shape memory polymer, may be released from their delivery configuration. Such release may permit, for example, anchors 40 to extend generally perpendicularly to access catheter 20 and to interatrial septum 15.

As illustrated in FIG. 7, access catheter 20 may then be withdrawn slightly, until anchors 40 are secured against, or generally abut, left atrial wall of interatrial septum 15, within left atrium 16 (not pictured in FIG. 7).

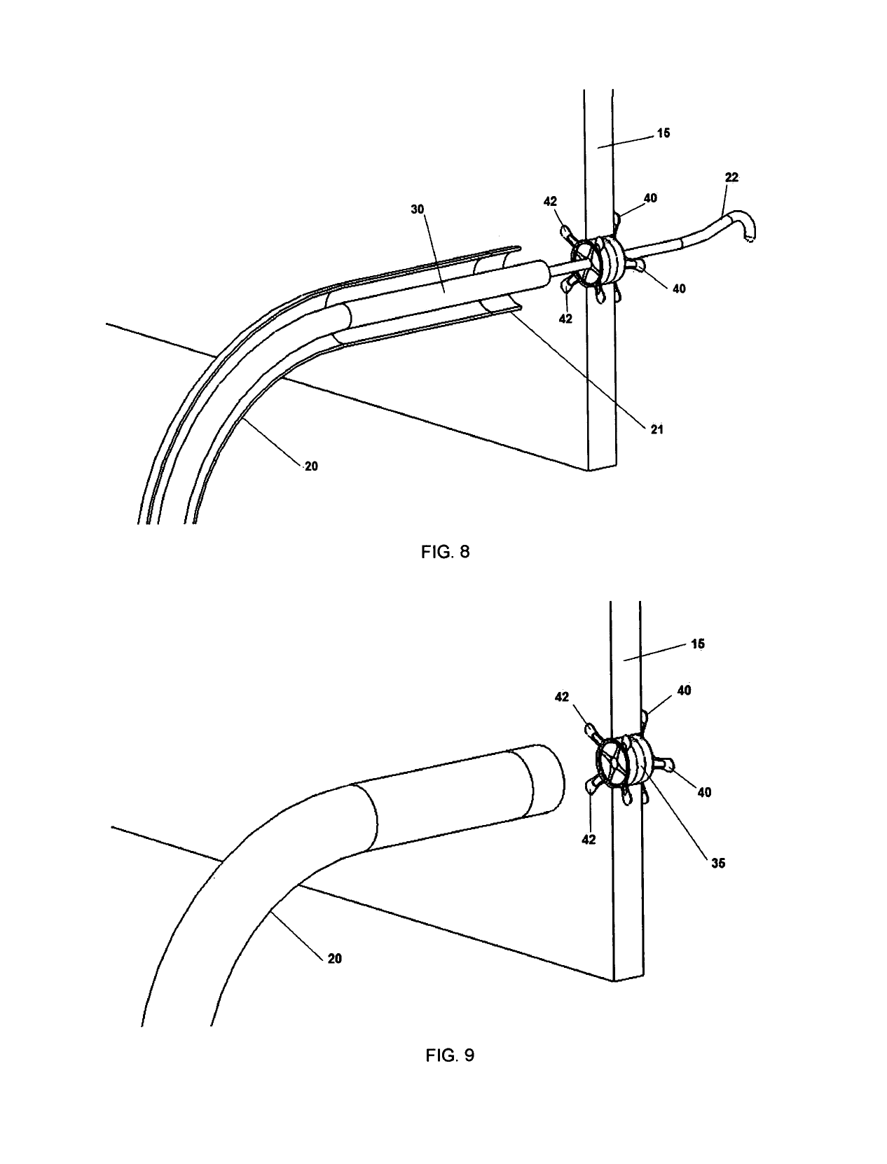

With first end anchors 40 securing implant 35 against interatrial septum 15, as illustrated in FIG. 8, distal end 21 of access catheter 20 and delivery catheter 30 have been withdrawn slightly further in order to release second end anchors 42 within right atrium 14 (not pictured in FIG. 8). Anchors 42 are now permitted to convert to their deployment configuration, and secure implant 35 to interatrial septum 15, from the right atrium side. Anchors 40 and 42 now secure interatrial septum 15 from opposite sides of septum 15 and hold implant 35 in place.

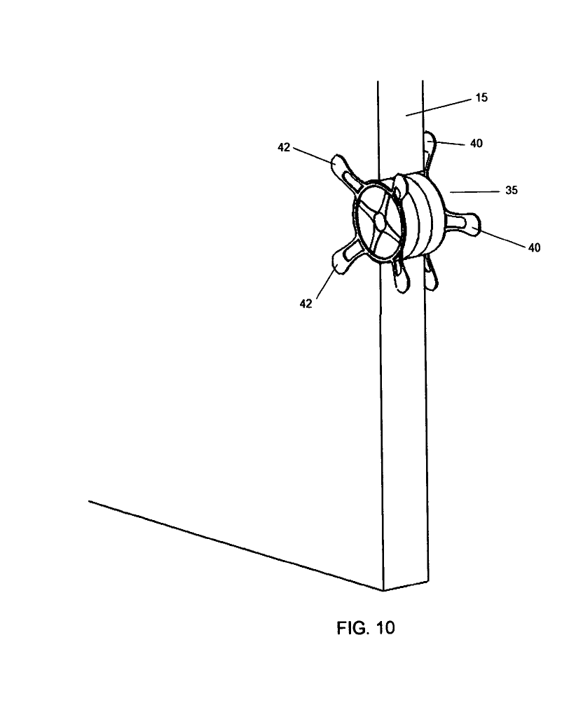

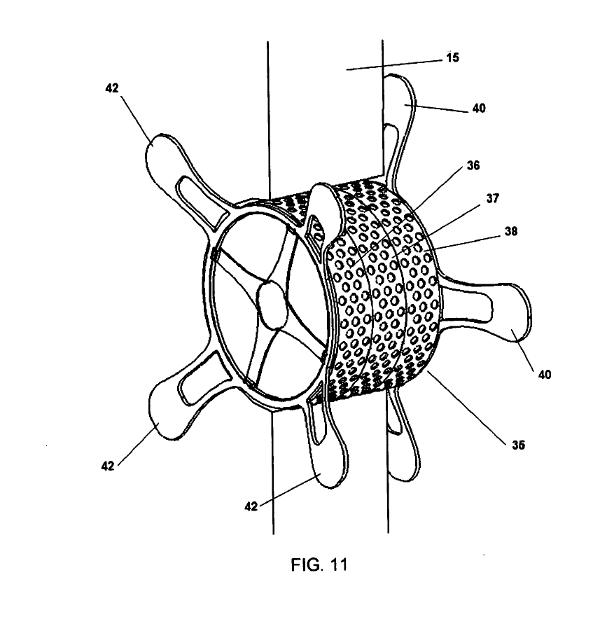

FIG. 9 illustrates in perspective view implant 35 deployed within atrial septal wall 15, subsequent to the withdrawal of guide 22, and during the withdrawal of access catheter 20, which is eventually complete, as shown in FIG. 10. FIG. 10 illustrates implant 35 following deployment and withdrawal of means for access and deployment.

FIG. 11 illustrates in larger detail implant 35 in its deployed configuration within interatrial septum 15. Implant 35 and anchors 40 and 42 may be reversibly deployable, allowing removal of implant 35 from a subject in a minimally invasive manner. Further, therapeutic scaffolds 36, 37, and 38 are removable from implant 35, allowing refilling or replacement of scaffolds. If scaffolds 36, 37 or 38 comprise viable tissue prepared to impart a pacemaker function, the tissue is in direct contact with the atrial septal wall of the subject, most directly along the sides of scaffolds 36, 37 or 38 (not visible in FIG. 11).

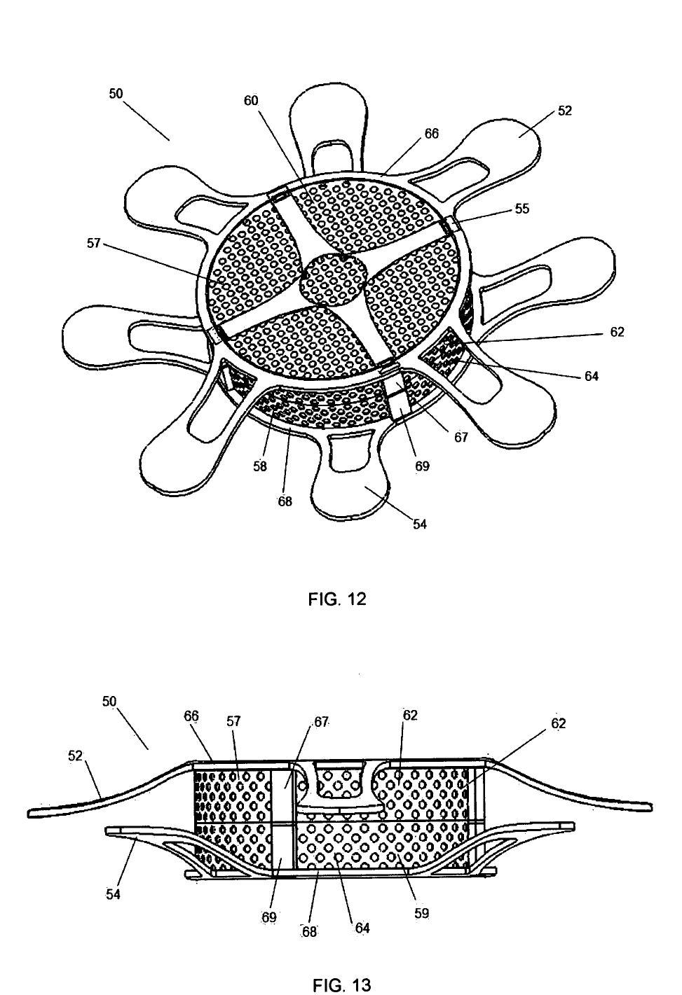

Turning now to an alternative embodiment according to the invention, FIG. 12 illustrates, in perspective view, implant 50 in a deployed configuration. Implant 50 may be delivered percutaneously in a delivery configuration (not shown) via a procedure similar to that described above in relation to FIGS. 4-11. For example, implant 50 may be delivered via a catheter or catheters through an incision to access the femoral vein, through the femoral vein to the inferior vena cava and ultimately to the right atrium and septal wall therein. Implant 50 may alternatively be delivered to the ventricular septal wall or other desired treatment or target site.

Implant 50 comprises first end anchors 52 which are integral with or affixed to first end frame 66, and second end anchors 54 which are integral with or affixed to second end frame 68. First end frame 66 and second end frame 68 are generally circular, and anchors 52 and 54 are generally evenly spaced about the circular structure, but alternative configurations may be suitable according to the invention. First end frame 66 and second end frame 68 generally secure first tissue scaffold 57 and second tissue scaffold 58.

Anchors 52 and 54 may be reversibly deployable, allowing release of the device from the atrial septal wall or the ventricular septal wall and retrieval via catheter. Accordingly, implant 50 may be removed from a subject. Further, scaffolds 57 and 58 may be transvascularly refillable or exchangeable from implant 50, allowing replacement of either or both scaffolds within frame 66.

Implant 50 further comprises scaffold connector 55, first scaffold top 60, first and second scaffold sides 62 and 64, which in this example are not covered by membrane. Following deployment of the device in a subject, scaffold sides 62 and 64 will be in direct contact with the septal wall of the subject. If scaffolds 57 and 58 comprise viable tissue prepared to impart a pacemaker function, the tissue's cells will be permitted to form gap junctions with the native cells of the subject. Scaffold sides 62 and 64 may alternatively be of a “scalloped”, comprise projections, or be of other irregular shape in order to increase the exposed surface area of first therapeutic scaffold 57 and second therapeutic scaffold. Greater surface area will potentially increase exposure of scaffolds 57 and 58 to contact with the native tissue of the interatrial septum of the subject in which the device will be implanted.

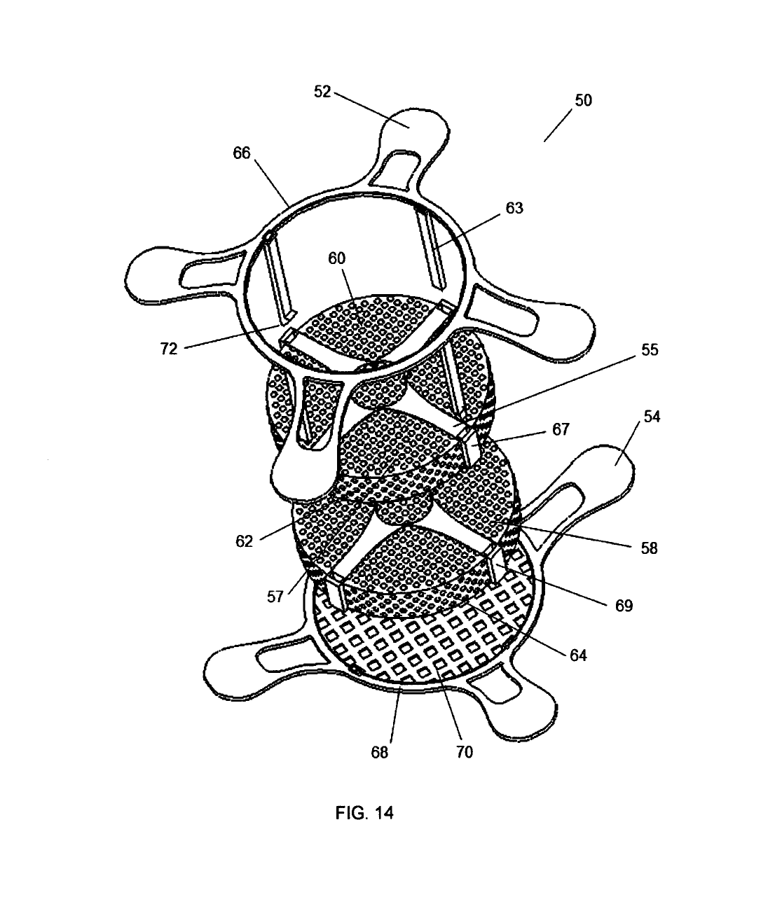

Also in the alternative, an implant may comprise only one therapeutic scaffold, or more than two therapeutic scaffolds. It also may comprise, in the alternative, a membrane covering all or a portion of the device, or one or both ends, as discussed in greater detail below in relation to FIG. 14.

Therapeutic scaffolds 57 and 58, retained by one or more optional scaffold connectors 55, have been prepared via suitable means discussed above to deliver a desired therapeutic agent. Following preparation according to suitable methods the scaffolds 57 and 58 are loaded into frames 66 and 68, and secured by one or more connectors 55. Therapeutic scaffolds 57 and 58 may be of any suitable size and dimension for delivery and retention at the target site within a subject.

When, for example, therapeutic scaffolds 57 and 58 comprise viable tissue which is capable of expressing a pacemaker gene, cell growth and expression of a pacemaker gene occurs within tissue scaffolds 57 and 58, which, in conjunction with frames 66 and 68 prevent undesirable migration of the cells. Electrical current is conducted from the isolated tissue in scaffolds 57 and 58, to the endogenous cardiac myocytes and throughout the heart in order to augment or restore lost pacemaker function of the heart, first in proximity to the natural AV (or SA) node. Cell growth and expression of a pacemaker gene occurs within tissue scaffolds 57 and 58, which together with frames 66 and 68, and anchors 52 and 54, prevent migration of scaffolds 57 and 58. Electrical current is conducted from scaffolds 57 and 58 to the endogenous cardiac myocytes and throughout the heart via cell-cell gap junction formation, phase change or other suitable mechanism in order to augment or restore lost pacemaker function of the heart, first in proximity to the natural AV node. Alternatively, scaffolds may comprise another therapeutic agent for which intraseptal delivery is desired.

The foregoing features are further illustrated in a side view of implant 50 in FIG. 13. First end anchors 52 are integral with or affixed to first end frame 66, and second end anchors 54 are integral with or affixed to second end frame 68. Scaffolds 57 and 58, retained by one or more optional scaffold connectors 55 (not visible in FIG. 13), are further secured by first and second mating slots 67 and 69. In this embodiment, scaffold sides 62 and 64 are not covered by membrane, and when deployed within a subject are permitted direct contact with the native tissue of the septal wall of the subject.

The assembly of implant 50 may be more clearly understood through a description of FIG. 14, which is an “exploded” view of the device. Frame 66 comprises optional alignment rails 63, which mate with optional mating slots 67 and 69. Rails 63 may further comprise locking tabs or other suitable means for securing rails 63 to frame 68. Though not pictured in FIG. 14, frame 68 may further comprise additional locking slots or other suitable means for further securing alignment rails 63.

Also as illustrated in FIG. 14, a device according to the invention may further comprise optional membrane 70 at one or both ends, or completely enclosing one or more therapeutic scaffolds. Further, portions of the membrane may comprise varied porosity and/or selective permeability in order to maximize the function of the particular portion of membrane. Membrane 70 is specially designed to comprise pores (not shown) of sufficient size to allow nutrient and metabolite transfer between the cells and the blood. Such nutrients and metabolites include, for example, oxygen, nitrogen, carbon dioxide, and lactic acid. The cells are exposed to oxygenated blood of the left atrium. The pores also permit a neurohormonal interface and exchange between the implanted cells and the blood of the subject. The pores however are too small to allow either cell migration or escape or to permit the entrance of cells or antibodies. Such pores are generally between approximately 0.1 micrometer and 10 micrometers in diameter, and sized to allow passage of molecules of a molecular weight of approximately 100,000 or less. In the alternative, membrane 70 may be selectively permeable according to the desired parameters for release and/or erosion of the particular therapy being delivered.

Membrane 70 is generally less than or equal to approximately 100 micrometers in thickness. The structure of the surface of the membrane may be varied to allow for strength and increased surface area for increased oxygen contact by adding composite fibers into the membrane wall or modifying the surface structure of the membrane. Portions of the membrane exposed to blood interface may be, for example, designed to maximize nutrient transfer, or, in the alternative, to regulate rate of therapy release. Further, the membrane may comprise, for example, porous ePTFE, or a membrane prepared according to any suitable nanopore membrane technology, including, but not limited to, stereolithography or soft lithography. The outer membrane may further be treated to either prevent cell growth on the exterior of implant 50, or, alternatively, to enhance cell growth and neovascularization, or otherwise comprise one or more therapeutic agents.

Analogous devices to induce or enhance muscle contraction in areas other than the heart are possible for the treatment of for example, obesity, stress incontinence, and other disorders. Such devices may be used in relation to stomach, esophageal, uterine, ureteral, urethral, bladder, jejunum or ileum smooth muscle cells.

While particular forms of the invention have been illustrated and described above, the foregoing descriptions are intended as examples, and to one skilled in the art it will be apparent that various modifications can be made without departing from the spirit and scope of the invention.

We claim:

1. An implantable device for the delivery and maintenance of a therapeutic scaffold, said device comprising one or more anchors, whereby said one or more anchors is configured to secure said device within a septal wall of the heart of a subject.

2. The device according to claim 1 wherein said therapeutic scaffold comprises a viable tissue prepared to impart a pacemaker function to the heart of a subject.

3. The device according to claim 1 wherein said therapeutic scaffold comprises one or more pharmaceutical, chemical, biological or radiological agents.

4. The device according to claim 1 further comprising a frame configured to retain said therapeutic scaffold.

5. The device according to claim 1 wherein said device may be delivered percutaneously to a subject.

6. The device according to claim 1 further comprising a delivery configuration and a deployed configuration.

7. The device according to claim 1 further comprising a selectively permeable membrane.

8. The device according to claim 1 wherein said device comprises one or more shape memory materials.

9. The device according to claim 1 wherein said one or more therapeutic scaffolds comprises one or more projections thereby increasing the surface area of the scaffold and the exposure of the scaffold to the cells of the septal wall of a subject.

10. The device according to claim 1 wherein said device is retrievably implantable.

11. The device according to claim 1 wherein said one or more therapeutic scaffolds is exchangeable.

12. An implantable device for delivering one or more viable, electrically conductive tissue scaffolds into a subject to induce or enhance muscle contraction.

13. A method for the minimally invasive treatment of a disease or condition comprising the steps of:
providing a device comprising one or more therapeutic scaffolds, said device comprising a delivery configuration and a deployed configuration;
accessing the right or left atrium, or right or left right ventricle of a subject;
penetrating the atrial or ventricular septal wall;
delivering the device to the atrial or ventricular septal wall; and
deploying the device within the atrial or ventricular septal wall.

14. The method according to claim 13 wherein said device comprises one or more anchors, with the added step of deploying the one or more anchors for securing the chamber within the atrial or ventricular septal wall.

15. The method according to claim 13 wherein said therapeutic scaffold comprises a viable tissue prepared to impart a pacemaker function to the heart of a subject.

16. The method according to claim 13 wherein said therapeutic scaffold comprises one or more pharmaceutical, chemical, biological or radiological agents.

17. The method according to claim 13 wherein said device is configured to retain said therapeutic scaffold.

18. The method according to claim 13 wherein said device comprises a selectively permeable membrane.

19. The method according to claim 13 wherein said device comprises one or more shape memory materials.

20. The method according to claim 13 wherein said device is retrievably implantable.

21. The method according to claim 13 wherein said one or more therapeutic scaffolds is exchangeable.

22. The method according to claim 13 wherein said one or more therapeutic scaffolds comprises viable, electrically conductive tissue to induce or enhance muscle contraction in a subject.

23. The method according to claim 13 wherein said method is used to treat a cardiac rhythm disorder.

24. The method according to claim 13 wherein the step of accessing the right or left atrium or right or left ventricle is performed percutaneously.
